(19) United States
(12) Patent Application Publication
(10) Pub. No.: US 2011/0306763 A1
(43) Pub. Date: Dec. 15, 2011

(54) PROCESS FOR THE PREPARATION OF IMATINIB AND SALTS THEREOF

(75) Inventors: Ajit Annu Kamath, Mumbai (IN); Ganesh Gurpur Pai, Mumbai (IN); Ashish Mohan Ujagare, Mumbai (IN); Xiao He, Shanghai (CN); Shaohong Wu, Shanghai (CN); Xin Shen, Shanghai (CN); Jidong Yang, Shanghai (CN); Huaxing Zhan, Shanghai (CN)

(73) Assignees: SHANGHAI PARLING PHARMAtECH CO., LTD., Shanghai (CN); ARCH PHARMALABS LIMITED, Mumbai (IN)

(21) Appl. No.: 13/203,141

(22) PCT Filed: Nov. 18, 2010
(86) PCT No.: PCT/IN10/00752

§ 371 Date: Aug. 24, 2011

(30) Foreign Application Priority Data
Dec. 10, 2009 (IN) 2853/MUM/2009

(51) Int. Cl. C07D 401/14

(52) U.S. Cl. 544/295

(57) ABSTRACT

Disclosed herein is a process for preparation of imatinib free base which comprises condensing 4-Methyl-N-(4-pyridin-3-yl-pyrimidin-2-yl)-benzene-1,3-diamine with 4-(4-Methyl-piperazin-1-ylmethyl)-benzoic acid ester in the presence of base including organic bases and inorganic bases in an organic solvent to form imatinib.

### TECHNICAL FIELD

The present invention relates to an improved process for the preparation of imatinib of the formula I comprising reaction between 4-Methyl-N-(4-pyridin-3-yl-pyrimidin-2-yl)-benzene-1,3-diamine of the formula II with 4-(4-Methyl-piperazin-1-ylmethyl)-benzoic acid derivatives of the formula III in the presence of a base.

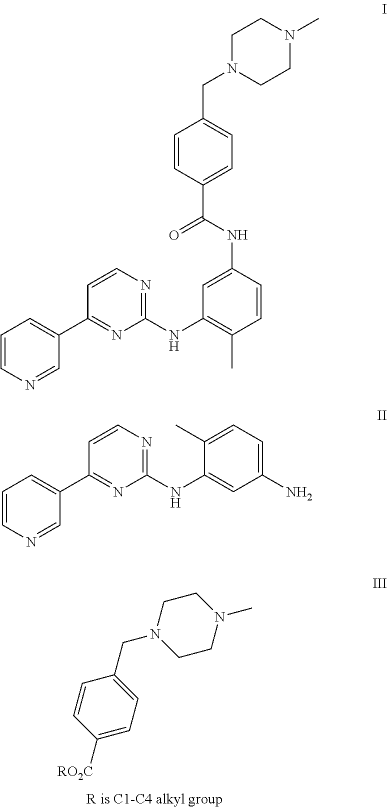

### BACKGROUND AND PRIOR ART

Imatinib is known as an inhibitor of protein-tyrosine kinase and is indicated for the treatment of chronic myeloid leukemia (CML). Imatinib also has potential for the treatment of various other cancers that express these kinase including acute lymphocyte leukemia and certain solid tumors. It can also be used for the treatment of atherosclerosis, thrombosis, restenosis, or fibrosis. Thus, imatinib can also be used for the treatment of non-malignant diseases. Imatinib is usually administered orally in the form of a suitable salt, e.g., in the form of imatinib mesylate.

The chemical name of Imatinib is 4-(4-methyl piperazine-1-methyl)-N4-methyl-3-[4-(3-pyridyl)pyrimidine-2-amino]-benzamide and is represented by the following structural formula:

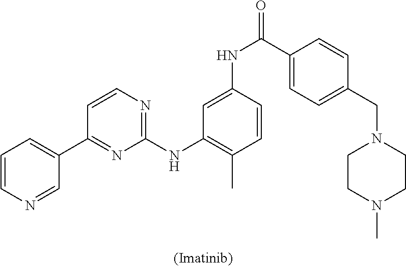

Imatinib Mesylate is an inhibitor of signal transduction (STI571) invented by Novartis AG after 7 years of hard work; it is the first inhibitor of cancer signal transduction ratified in the whole world. It is sold by Novartis as Gleevec capsules containing imatinib mesylate in amounts equivalent to 100 mg or 400 mg of imatinib free base.

Imatinib Mesylate is the rare drug in America, European Union and Japan. In May 10, 2001, it was ratified by American Food and Drug Administration (FDA) to treat the chronic myelogenous leukemia patients.

EP0564409 (U.S. Pat. No. 5,521,184) describes the process for the preparation of imatinib and the use thereof, especially as an anti tumour agent.

There are generally two synthetic routes for synthesis of Imatinib, suitable for the industrial production. One synthetic process as described in scheme-I comprises using 2-methyl-5-nitroaniline as the raw material which is reacted with cyanamide to obtain guanidine; cyclization reaction with 3-dimethylamino-1-(3-pyridyl)-2-propylene-1-ketone; reduction step of nitro to amine and condensation reaction with 4-(Chloromethyl)benzoyl chloride and N-methylpiperazidine to obtain Imatinib (WO 2004/108669).

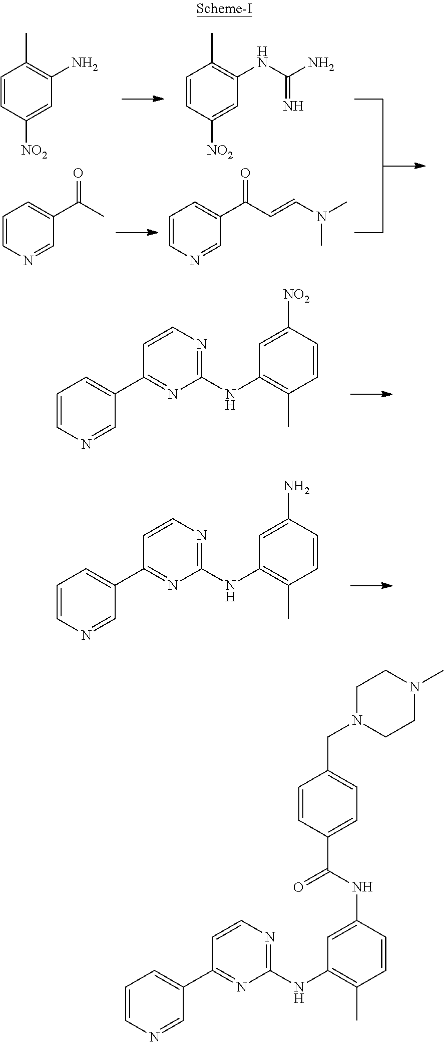

Scheme-2 describes the successful process for the synthesis of Imatinib using 4-methyl-3-nitroanilines as the raw material, comprising reacting 4-methyl-3-nitroanilines with 4-(Chloromethyl)benzoyl chloride and N-methyl piperazidine in turns; followed by reduction of nitro group to amino group; then reaction with cyanamide to obtain guanidine; finally cyclization reaction with 3-dimethyl amino-1-(3-pyridyl)-2-propylene-1-ketone to obtain Imatinib (WO 03/066613). The said PCT application discloses the use of 4-4-(methyl piperazin-1-ylmethyl)-benzoic acid methyl ester as one of the raw material but rest of the reactants are different from that of N-(5-amino-2-methylphenyl)-4(3-pyridyl)-2-pyrimidine amine in presence of trimethyl aluminium.

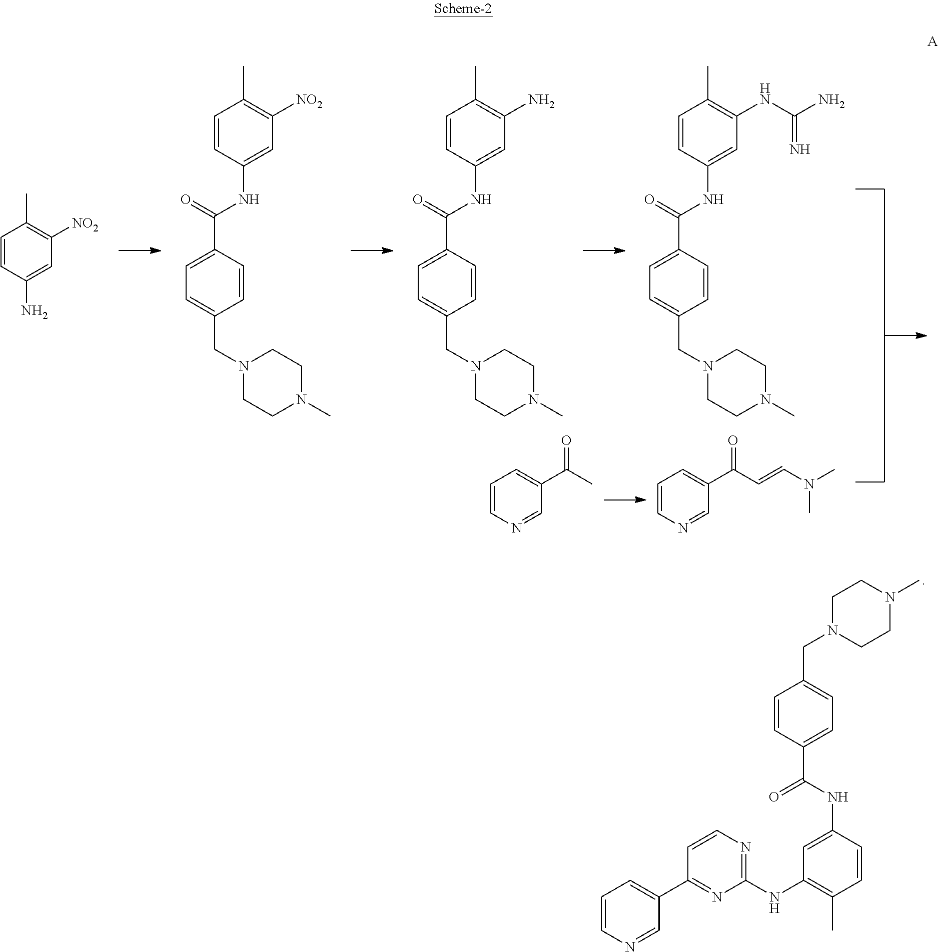

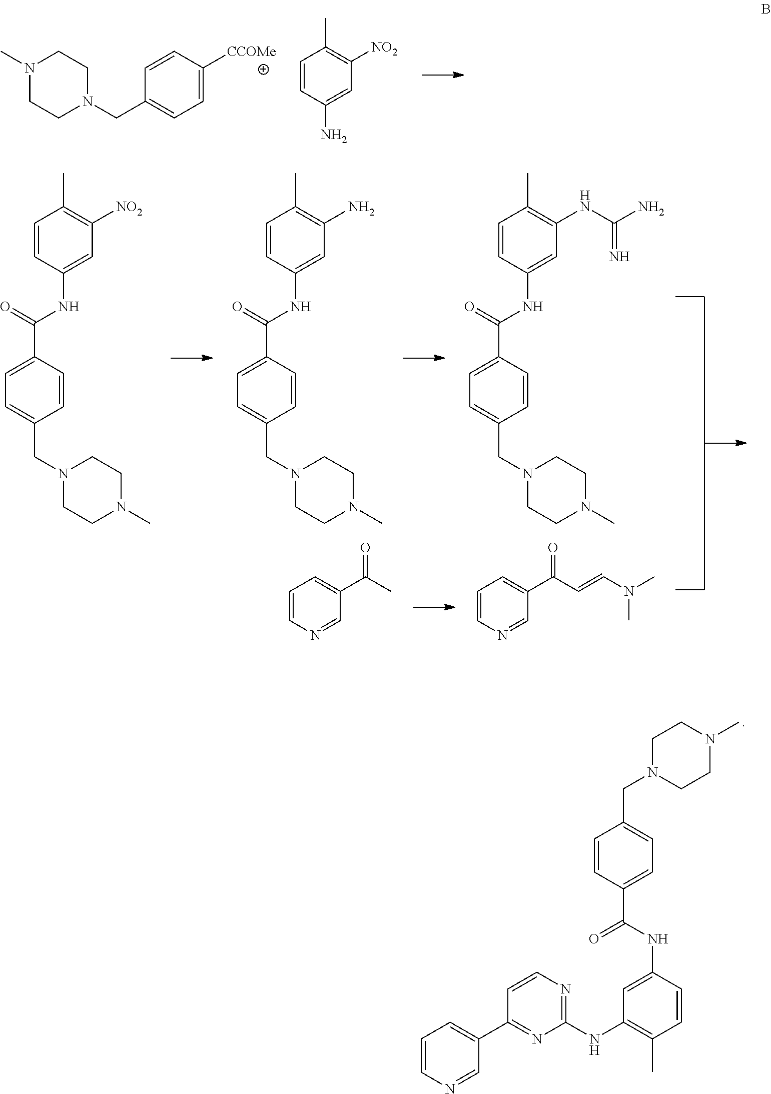

Common feature of the processes for preparing imatinib according to (WO 2004/108669) and (WO03/066613) lies in use of cyanamide as a reagent. The main difference between the two routes is that the reaction sequence of cyclization of pyrimidine chain is different.

Example 10 of PCT International Publication no. WO 2003/066613 is less applicable to industrial purposes. These include the reaction between N-(3-bromo-4-methyl-phenyl)-4-(4-methyl-piperazin-1-ylmethyl)-benzamide and 4-(3-pyridyl)-2-pyrimidineamine which uses a mixture of rac-BINAP (a phosphine oxide catalyst) and Pd₂ (dba)₃*CHCl₃. These catalysts are very expensive, therefore, their use is unfit for commercial production.

CN1630648A describes a process comprising reaction of 3-bromine-4-methyl aniline with 4-(4-methyl-piperazin-methyl)methyl benzoate in presence of trimethyl-Aluminum to obtain N-(4-methyl-3-bromobenzene)-4-(4-methyl-piperazin-1-methyl)-benzamide, which further reacts with 2-amino-4-(3-pyridyl)-pyrimidine in presence of palladium as catalyst to obtain Imatinib.

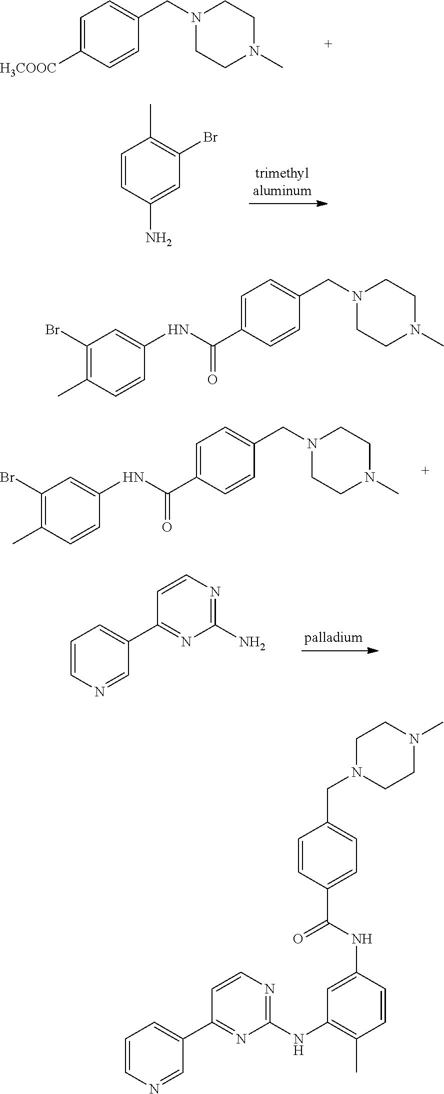

The drawback of the above process is the use of trimethyl-Aluminum, which is flammable and reacts severely when comes in contact with water.

CN101016293A describes another process using N-(4-methyl-3-3-aminophenyl)-4-(4-methyl-piperazin-1-methyl)-benzamide as the raw material. The said raw material is reacted with 2-halogen-4-(3-pyridyl)-pyrimidine to obtain Imatinib.

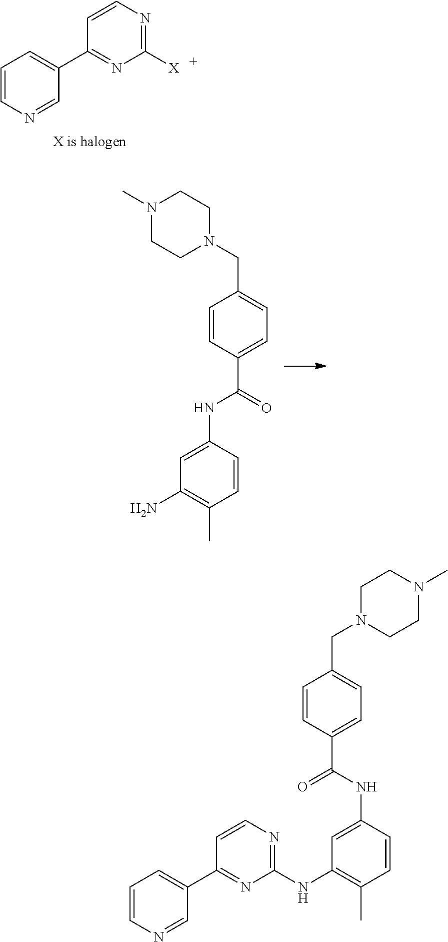

The process disclosed in CN101016293A comprises use of halogenated agent, such as phosphorus oxychloride, which is used to synthesize 2-halogeno-4-methyl-(3-pyridyl)-pyridine is lachrymator and corrosive and has great influence to the surroundings. EP0564409 describes a coupling reaction between N-(5-amino-2-methylphenyl)-4-(3-pyridyl)-2-pyrimidine amine and 4-(4-methyl piperazin-1-ylmethyl)-benzoyl chloride in the presence of high quantity of pyridine to starting reactant amine N-(5-amino-2-methylphenyl)-4-(3-pyridyl)-2-pyrimidine amine. The ratio of the pyridine to the said reactant is 138 which is equivalent to about 40 parts v/w. Use of such a large quantity of pyridine is unsafe as it is a toxic solvent according to ICH guidelines. The workup of the reaction comprises evaporation of the remaining pyridine and further processing, which finally involves chromatography for purification, which is highly undesirable on industrial scale because it is expensive and time consuming

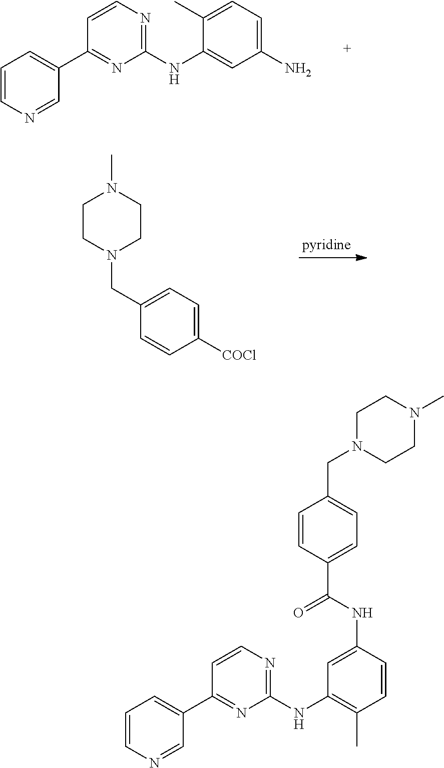

US2006/0149061 and US20060223817 also discloses a similar synthetic approach comprising the use of similar pyridine/starting amine ratio (140 equivalents which is equals about 41 parts v/w). The product obtained is purified by slurring in ethyl acetate.

WO2004/074502 describes a coupling reaction between N-(5-amino-2-methylphenyl)-4-(3-pyridyl)-2-pyrimidine amine and 4-(4-methyl piperazin-1-ylmethyl)-benzoyl chloride wherein solvent like dimethyl pharmamide, dimethyl acetamide, N-methylpyrilidinone are used as solvents instead of pyridine. However the method described in this patent application lacks an advantage in that the coupling reaction produces the hydrohalide salt of imatinib, e.g. imatinib trihydrochloride monohydrate, which has to be treated with a base in order to afford the imatinib base, thus an extra step is required. Further, conventional methods for coupling N-(5-amino-2-methylphenyl)-4-(3-pyridyl)-2-pyrimidine amine require reaction with an acid halide, e.g. 4-(4-methyl piperazin-1-ylmethyl)-benzoyl chloride, which requires an additional production step that can involve harsh and/or toxic chlorinating agent.

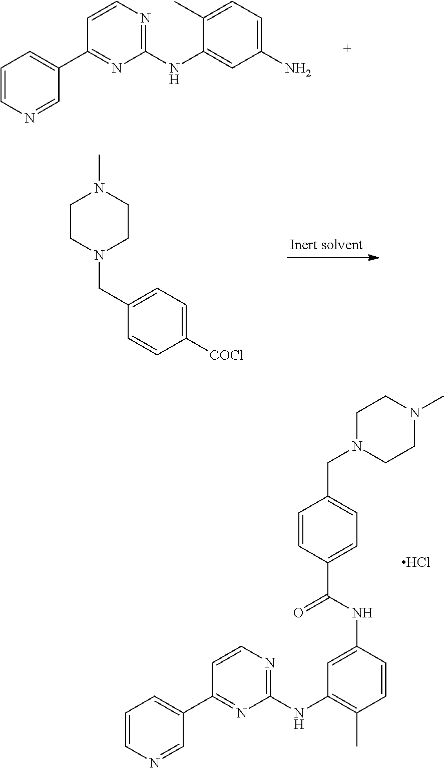

WO2008/117298 describes a coupling reaction between N-(5-amino-2-methylphenyl)-4-(3-pyridyl)-2-pyrimidine amine and 4-(4-methyl piperazin-1-ylmethyl)-benzoyl chloride in presence of a base selected from potassium carbonate, sodium carbonate, potassium or sodium hydroxide. Use of potassium carbonate as base results into the formation of Imatinib dihydrochloride which ultimately requires an additional operation of neutralization by using excessive base to get imatinib.

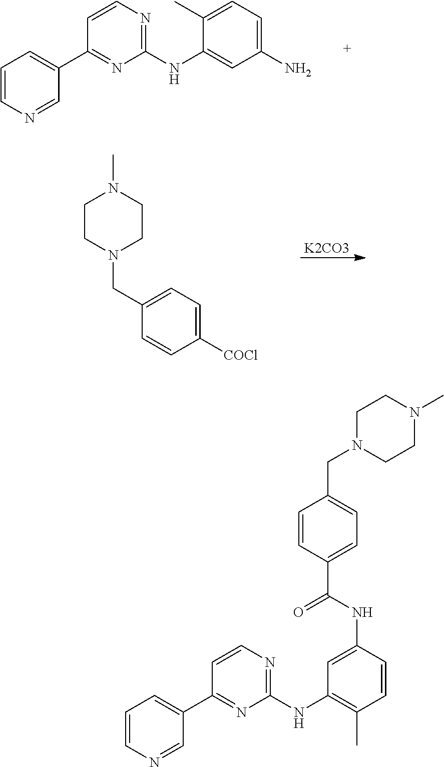

WO2008/136010 describes a coupling reaction between N-(5-amino-2-methylphenyl)-4-(3-pyridyl)-2-pyrimidine amine and 4-(4-methyl piperazin-1-ylmethyl)-benzoyl chloride in presence of base potassium hydroxide resulting into 78.6% yield of crude imatinib base. Preparation of crude requires imatinib hydrochloride preparation during the workup which is then basified to get base in crude form. This also describes maleate salt preparation as mode of purification which is again basified to give pure Imatinib base.

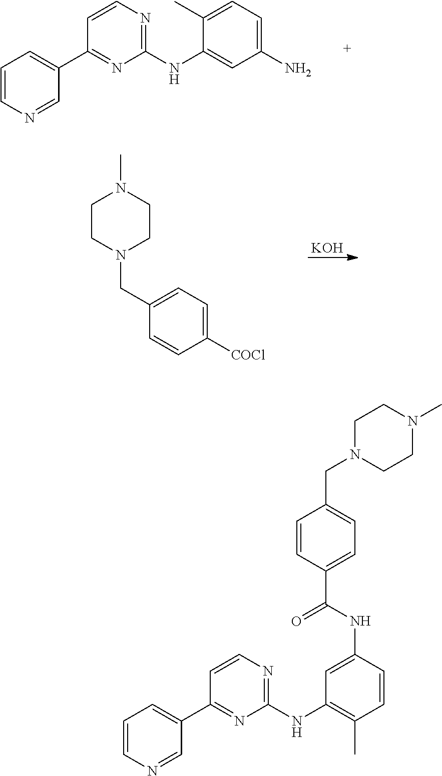

US patent application 2004/0248918 discloses a different approach using N-(5-amino-2-methylphenyl)-4-(3-pyridyl)-2-pyrimidine amine and 4-(2-chloromethyl)-benzoyl chloride as raw material. The reaction for the preparation of Imatinib is carried out in tetrahydrofuran as a reaction solvent and in the presence of pyridine as a base. However the method described in this patent application lacks an advantage as purification of the product requires column chromatography using chloroform:methanol (3:1 v/v), which is not suitable purification method when performing the reaction on large scale, followed by crystallization.

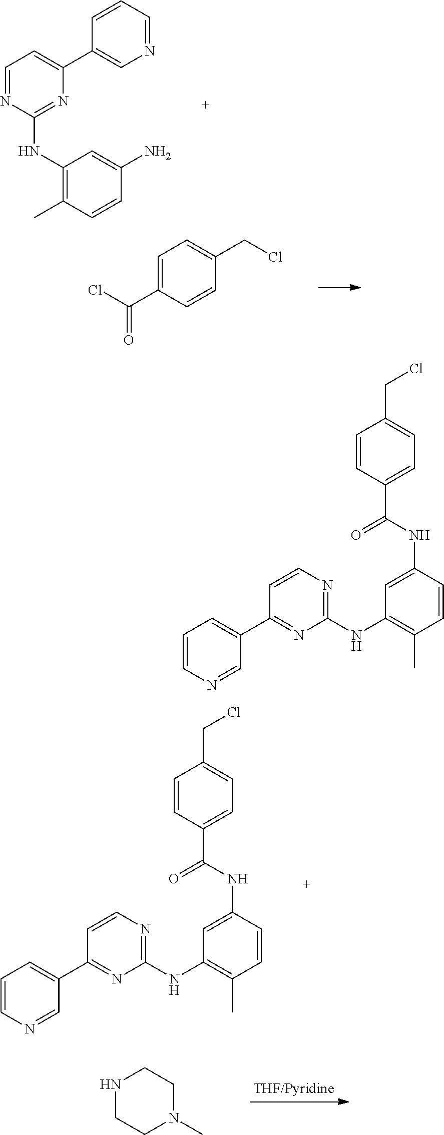

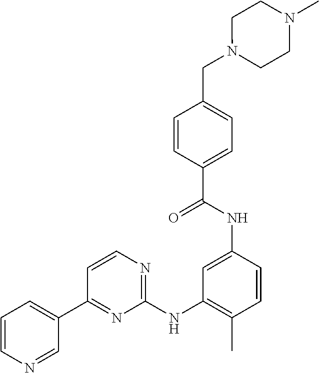

US patent application 2008/0103305 discloses a process comprising reacting N-(5-amino-2-methylphenyl)-4-(3-pyridyl)-2-pyrimidine amine or its alkyl derivative and an acid salt of 4-[(4-methyl-1-piperazinyl)-methyl]benzoyl derivative as given below in the scheme-3 using pyridine in an amount of about 2 to 10 volumes per gram of the said amine. However the drawback associated with this process is use of pyridine especially when reaction is performed on large scale.

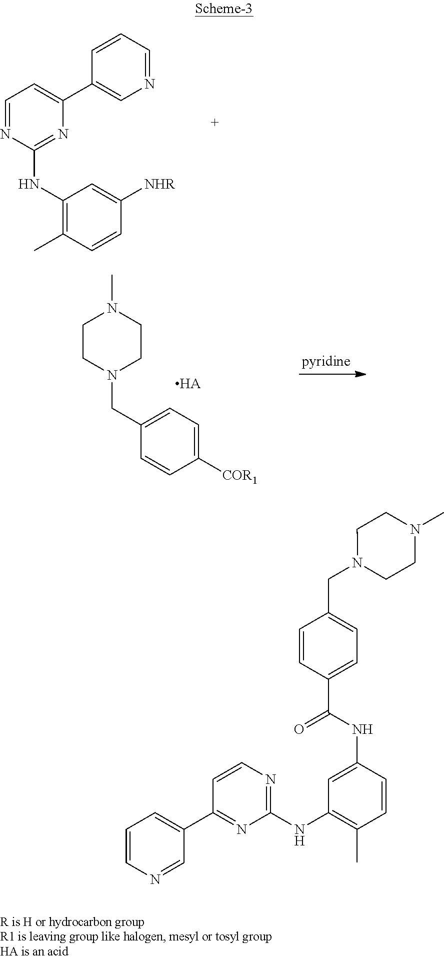

Thus, there is a need in the art to develop an efficient and safe synthetic route which overcomes the limitations in the processes disclosed in the prior art cited hereinabove. Further, there is a need in the art for a process for preparing imatinib which is less hazardous and more environmental friendly and is suitable for scale up, does not require the use of large quantities of pyridine and does not use the chromatography as a means of purification, avoiding the use of hazardous 4-(4-methyl piperazin-1-ylmethyl)-benzoyl chloride as raw material which in turn is hazardous for the preparation and also does not require formation of salts for the purification to remove impurities. Moreover, avoid formation of corrosive byproduct when acyl chloride is used as raw material. Though the prior art has shown the use of ester of 4-(4-Methyl-piperazin-1-ylmethyl)-benzoic acid, but there is an intention of making the nitro compound which is then further reduced to get amino intermediate.

Advantages of the present invention over the prior art:
1) Use of safe and easy to prepare esters of 4-(4-Methyl-piperazin-1-ylmethyl)-benzoic acid as a starting material instead of hazardous acid chloride.
2) Byproducts are simple alcohols in place of corrosive hydrogen chloride
3) Avoids the use of large quantity of obnoxious and foul smelling pyridine as solvent and its tedious work up.
4) Avoids extra operation of salt preparation as mode of purification.
5) Column chromatography is necessary ti isolate the product in pure form and column chromatography becomes unpractical on commercial scale.

### OBJECT OF THE INVENTION

The object of the present invention is to develop a simple, safe and efficient process for the preparation of substantially pure imatinib base and salt thereof.

The object of the present invention is to provide a process for the coupling reaction between the ester of 4-(4-Methyl-piperazin-1-ylmethyl)-benzoic acid replacing corresponding hazardous acid chloride and N-(5-amino-2-methylphenyl)-4-(3-pyridyl)-2-pyrimidine amine.

Another aspect of the present invention is to provide the simple bases those can be used for the coupling of 4-(4-Methyl-piperazin-1-ylmethyl)-benzoic acid ester and N-(5-amino-2-methylphenyl)-4-(3-pyridyl)-2-pyrimidine amine.

It is an object of the invention to provide a simple process for preparation of imatinib by condensing 4-Methyl-N-(4-pyridin-3-yl-pyrimidin-2-yl)-benzene-1,3-diamine with 4-(4-Methyl-piperazin-1-ylmethyl)-benzoic acid ester in a suitable organic solvent.

### SUMMARY OF THE INVENTION

The present invention discloses a new and efficient process for the preparation of imatinib comprising reacting 4-Methyl-N-(4-pyridin-3-yl-pyrimidin-2-yl)-benzene-1,3-diamine with 4-(4-Methyl-piperazin-1-ylmethyl)-benzoic acid ester replacing hazardous-4-(4-Methyl-piperazin-1-ylmethyl)-benzoic acid chloride in a suitable solvent and simple base to yield substantially pure imatinib base in about 90% yield.

### DETAILED DESCRIPTION OF THE INVENTION

In an embodiment of the present invention, there is provided a process for the preparation of imatinib, which comprises the reaction of 4-Methyl-N-(4-pyridin-3-yl-pyrimidin-2-yl)-benzene-1,3-diamine (II) also referred as N-(5-amino-2-methylphenyl)-4-(3-pyridyl)-2-pyrimidine amine with 4-(4-Methyl-piperazin-1-ylmethyl)-benzoic acid ester (III) in the presence of a base in a suitable solvent to yield substantially pure imatinib base in about 90% yield.

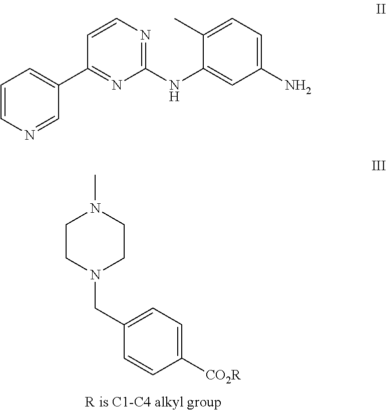

The preparation of 4-Methyl-N-(4-pyridin-3-yl-pyrimidin-2-yl)-benzene-1,3-diamine (II) and 4-(4-Methyl-piperazin-1-ylmethyl)-benzoic acid ester (III) may be carried out according to prior art methods.

Compound of formula (II) can be synthesized by the process disclosed in WO2004/108669 comprising

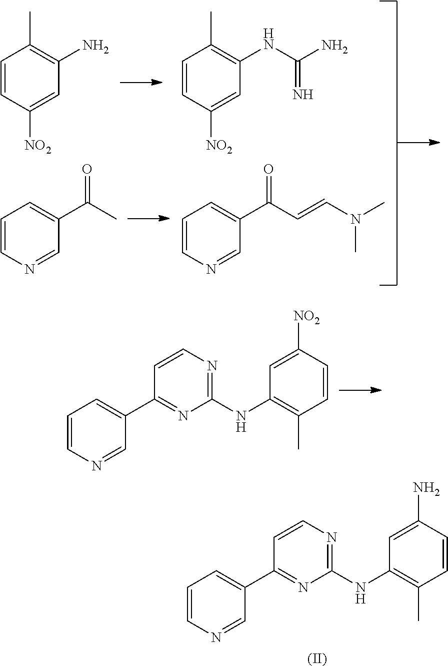

reacting 2-methyl-5-nitroaniline with 50% aqueous solution of cyanamide to obtain N-(2-Methyl-5-nitrophenyl)-guanidinium nitrate, which further reacted with 3-dimethylamino-1-pyridin-3-yl-propenone to yield (2-methyl-5-nitrophenyl)-(4-pyridin-3-yl-pyrimidin-2-yl)-amine, finally, reduction of nitro group to obtain compound of formula (II).

Compounds of formula (III) can be synthesized by the process disclosed in synthtic communications 2003, 3597

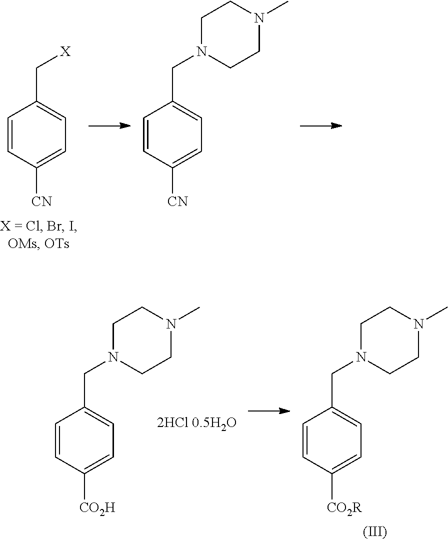

comprising reacting α-halogen-p-toluinitrile or methanesulfonic acid 4-cyano-benzyl ester or toluene-4-sulfonic acid 4-cyano-benzyl ester with N-methylpiperazine, followed by hydrolysis of the cyano to acid which formed as dihydrochloride contain half crystalline hydrate, finally reaction with alcohol to obtain compound of formula (III).

The synthetic route for preparing imatinib according to the present invention is given below:

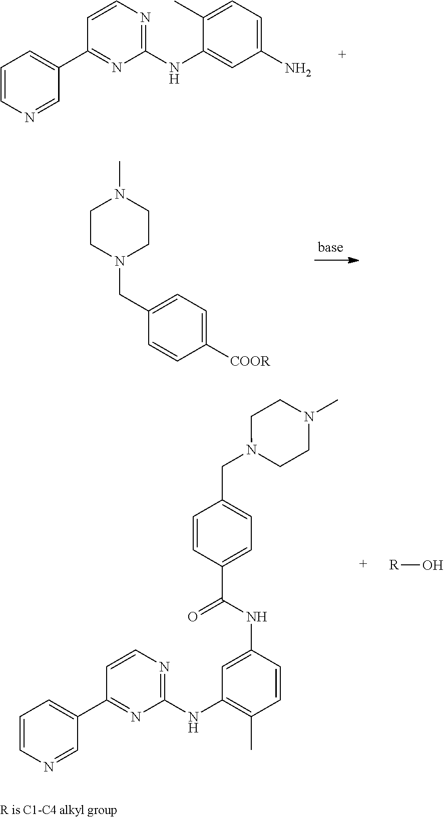

In a preferred embodiment of the present invention, the coupling reaction is carried out using base selected from organic bases such as sodium alkoxide (sodium methoxide, sodium ethoxde, sodium propoxide, sodium butoxide, sodium tert-butoxide), potassium alkoxide (potassium methylate, potassium ethylate, potassium propoxide, potassium butoxide, potassium tert-butoxide), butyllithium, s-butyllithium and tert-butyllithium; and, inorganic bases such as sodium hydroxide, potassium hydroxide, lithium hydroxide, cesium hydroxide and the like.

The concentration of base used in reaction solution is in the range from about 0.1M to about 10M.

The coupling reaction is carried at a reaction temperature ranging from about 20 to about 100° C. preferably ranging between about 25 to about 30° C.

The solvent is selected from straight chain or branched C₁-C₄ alcohols selected from methanol, ethanol, isopropyl alcohol and the like, ethers selected from tetrahydrofuran, diethyl ether, isopropyl ether and the like, chlorinated hydrocarbons selected from methylene chloride, 1,2-dichloroethane and the like, nitriles selected from acetonitrile and the like, hydrocarbons selected from toluene, dimethylbenzene and the like, esters selected from ethyl acetate and the like, polar aprotic solvents selected from dimethyl sulfoxide, dimethylfomamide and the like or mixture thereof.

The ester group of 4-(4-Methyl-piperazin-1-ylmethyl)-benzoic acid ester (II) is selected from C₁-C₄ straight chain or branched carbon alkyl ester such as methyl ester, ethyl ester, propyl ester, butyl ester, tert-butyl ester, pentyl ester; or substitutive benzyl ester, substituted phenyl ester and the like.

The present invention discloses a process comprising reacting 4-Methyl-N-(4-pyridin-3-yl-pyrimidin-2-yl)-benzene-1,3-diamine (II), with 4-(4-Methyl-piperazin-1-ylmethyl)-benzoic acid ester (III) in the presence of a base in a suitable solvent to obtain Imatinib.

The process of the present invention overcomes all the limitations cited hereinabove of the processes disclosed in prior art. The reaction is carried out under moderate conditions and is easy to operate. The aminolysis reaction of ester is easy and clean. Further, the by-product is alcohol which is nontoxic thereby, making the process eco-friendly. The product obtained is substantially pure which does not require salt formation to remove impurities and yield is high and is suitable for industrial production.

The following examples, which include preferred embodiments, will serve to illustrate the practice of this invention, it being understood that the particulars shown are by way of example and for purpose of illustrative discussion of preferred embodiments of the invention.

### EXAMPLES

### Example 1

To a solution of 4-Methyl-N-(4-pyridin-3-yl-pyrimidin-2-yl)-benzene-1,3-diamine (27.7 g) and 4-(4-Methyl-piperazin-1-ylmethyl)-benzoic acid methyl ester (50 g) in Tetrahydrofuran (250 ml), a solution of sodium methylate (10 g) in methanol (10 ml) was added. The reaction mixture was heated to reflux. After completion of the reaction solution was poured into ice-water and a large amount of solid precipitated, which was filtered and washed with water and dried to obtain Imatinib base (45 g). Yield: 91%.

The spectral data is as follows:

¹H NMR (500M, DMSO) δ: 10.2 (s, 1H), 9.30 (s, 1H), 8.99 (s, 1H), 8.72 (d, J=4.0 Hz, 1H), 8.57 (s, 1H), 8.53 (s, 1H), 8.11 (s, 1H), 8.00 (s, 1H), 7.98 (s, 1H), 7.58-7.51 (m, 4H), 7.44 (d, J=4.3 Hz, 1H), 7.22 (d, J=8.1 Hz, 1H), 3.70 (s, 2H), 3.50-3.25 (m, 2H), 3.20-2.90 (m, 4H), 2.81 (s, 3H), 2.40 (s, 3H), 2.24 (s, 3H). ¹³C NMR (125M, DMSO) δ: 164.9, 161.3, 161.1, 159.4, 150.8, 147.7, 137.7, 137.1, 134.9, 134.3, 132.3, 129.9, 129.1, 127.7, 127.6, 123.9, 117.2, 116.8, 107.5, 59.9, 52.1, 48.9, 42.2, 17.5. MS (M⁺+1): 494.3

### Example 2

To a solution of 4-Methyl-N-(4-pyridin-3-yl-pyrimidin-2-yl)-benzene-1,3-diamine (27.7 g) and 4-(4-Methyl-piperazin-1-ylmethyl)-benzoic acid methyl ester (50 g) in toluene (250 ml), a solution of sodium ethoxide (20 g) in methanol (10 ml) was added. The reaction mixture was heated to reflux. After completion of the reaction, solution was poured into ice-water and a large amount of solid precipitated, which was filtered and washed with water and dried to obtain Imatinib base (44 g). Yield: 91%.

### Example 3

To a solution of potassium butoxide (250 g) in methanol (1000 ml), a solution of 4-Methyl-N-(4-pyridin-3-yl-pyrimidin-2-yl)-benzene-1,3-diamine (277 g) and 4-(4-Methyl-piperazin-1-ylmethyl)-benzoic acid propyl ester (600 g) in Tetrahydrofuran (2500 ml) was added. The reaction mixture was stirred at room temperature. After completion of the reaction solution was poured into ice-water and a large amount of solid precipitated, which was filtered and washed with water and dried to obtain Imatinib base (450 g). Yield: 91%.

### Example 4

To a solution of potassium butoxide (25 kg) in ethanol (100 Litre), a solution of 4-Methyl-N-(4-pyridin-3-yl-pyrimidin-2-yl)-benzene-1,3-diamine (27.7 kg) and 4-(4-Methyl-piperazin-1-ylmethyl)-benzoic acid ethyl ester (50.0 kg) in toluene (250 Litre) was added. The reaction mixture was stirred at room temperature. After completion of reaction, solution was poured into ice-water and a large amount of solid precipitated, which was filtered and washed with water, and dried to obtain Imatinib base (40.0 kg). Yield: 81%.

1. A process for the preparation of substantially pure imatinib of formula I and acid addition salt thereof comprising reacting 4-Methyl-N-(4-pyridin-3-yl-pyrimidin-2-yl)-benzene-1,3-diamine of formula II with 4-(4-Methyl-piperazin-1-ylmethyl)-benzoic acid ester of formula III in the presence of an organic solvent and a base.

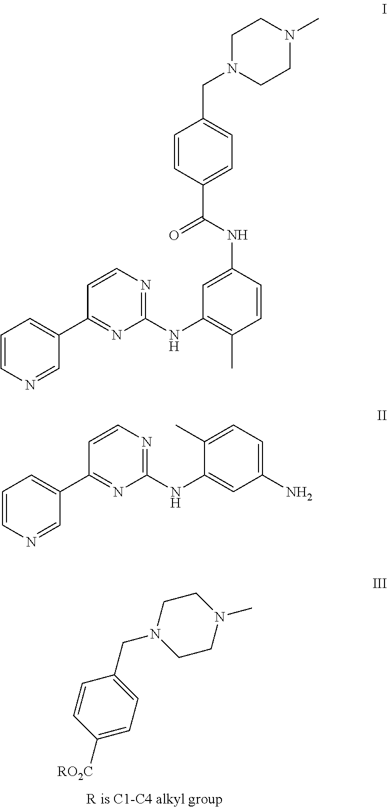

2. The process of claim 1 wherein base is selected from alkali metal alkoxide, alkyl lithium and metal hydroxide.

3. The process of claim 2 wherein alkali metal alkoxide is selected from sodium methoxide, sodium ethoxide, sodium propoxide, sodium butoxide, sodium tert-butoxide, potassium methylate, potassium ethylate, potassium propoxide, potassium butoxide, or potassium tert-butoxide.

4. The process of claim 2 wherein alkyl lithium is selected from butyllithium, s-butyllithium and tert-butyllithium.

5. The process of claim 2 wherein metal hydroxide is selected from sodium hydroxide, potassium hydroxide, lithium hydroxide, cesium hydroxide and the like.

6. The process according to claim 1, wherein the reaction is carried out at a temperature ranging from about 20 to about 100° C.° C.

7. The process of claim 1 wherein solvent is selected from alcohols selected from methanol, ethanol; ethers selected from tetrahydrofuran, diethyl ether, isopropyl ether; chlorinated hydrocarbons selected from methylene chloride, 1,2-dichloroethane; nitriles selected from acetonitrile; hydrocarbons selected from toluene, dimethylbenzene; esters selected from ethyl acetate; or polar aprotic solvents selected from dimethyl sulfoxide, dimethylfomamide.
